(19) United States
(12) Patent Application Publication
(10) Pub. No.: US 2023/0363414 A1
(43) Pub. Date: Nov. 16, 2023

(54) FOOD AND SUPPLEMENT FORMULATIONS

(71) Applicant: SUSTENTO INC., Mississauga (CA)

(72) Inventors: Sofia BONILLA, Mississauga (CA); Kasey DUNN, Toronto (CA)

(73) Assignee: SUSTENTO INC., Mississauga, ON (CA)

(21) Appl. No.: 18/028,882

(22) PCT Filed: Sep. 28, 2021
(86) PCT No.: PCT/CA2021/051345

§ 371 Date: Mar. 28, 2023

Related U.S. Application Data
(60) Provisional application No. 63/084,384, filed on Sep. 28, 2020.

(51) Int. Cl. A23K 10/30; A61K 35/64; A61K 36/05; A61K 36/04; A61K 36/064; A61K 36/48; A61K 36/03; A61K 36/55; A61K 36/39; A23K 20/174; A23K 10/20; A23K 40/30; A23K 50/40; A23K 20/142; A23K 20/158

(52) U.S. Cl. CPC A23K 10/30; A61K 35/64; A61K 36/05; A61K 36/04; A61K 36/064; A61K 36/48; A61K 36/03; A61K 36/55; A61K 36/39; A23K 20/174; A23K 10/20; A23K 40/30; A23K 50/40; A23K 20/142; A23K 20/158

(57) ABSTRACT

The present disclosure relates to a companion animal (e.g. pet) food and/or supplement formulations, and more particularly to a novel animal product or by-product -free, naturally based food and/or supplement formulation that has the nutritional profile of those made with animal products (e.g. meat based).

### CROSS-REFERENCE TO RELATED APPLICATIONS

The application is a National Phase Entry of International Patent Application No. PCT/CA2021/051345, filed on Sep. 28, 2021, which claims priority to U.S. Provisional Patent Application No. 63/084,384, filed Sep. 28, 2020, both of which are incorporated by reference herein.

### TECHNICAL FIELD

The present invention relates to food and/or dietary supplement formulations, and more particularly to an animal-free (e.g. meat-free), naturally based food and/or supplement formulation that has the nutritional profile of those made with animal products (e.g. meat based).

### BACKGROUND

Increasingly, people are seeking nutritional food and dietary formulations that contain no animal products. As such, there continues to be a need for non-animal based food formulations suitable for humans (both adults and infants), and animals, including pets such obligate carnivore pets.

Companion animals, such as, for example, dogs and cats, have certain nutritional and nutrient needs. While requiring different nutritional needs, fish also require feed or nutritional supplements which are typically derived from animal based products. Currently, these needs are being met only through animal-based products or synthetically produced supplements. For example, some pets, such as cats, lizards, and snakes, are generally regarded as “obligate carnivores” meaning that these pets cannot obtain their nutritional needs from animal-free diets. As such, there is a need for non-animal based pet food formulations suitable for such obligate carnivore pets.

Animal-based products and their production can also be seen as having negative impacts on the environment and may ultimately be unsustainable. Further, many pet owners and consumers want to have a sustainable source of nutrition for their pets and as such, want to detach or delink from traditional animal based farming industries. On the other hand, animal or meat-free pet food products may not provide key or essential nutrients in their natural form. Such key or essential nutrients must be added to the food as dietary supplements or given separately. Dietary supplements include vitamins, minerals, amino acids, fatty acids, herbs, botanicals, enzymes, and probiotics. Essential nutrients may be either added to pet food during production or sold separately as nutritional supplements. Many times, these supplements are synthetically (e.g. made through chemical or other processes) produced. Such synthetic supplements may not have the same nutritional properties as their natural counterpart and may not be produced in an environmentally friendly fashion. Without such supplements, however, the use of meat-free diets may result in nutrient deficiencies.

Foods made with animal or meat-based products contain taurine and arachidonic acid and, in some cases, these nutrients are added to the formula in the form of dietary supplements. Taurine is an amino acid nutritional supplement that is used to treat taurine-deficiency diseases such as dilated cardiomyopathy, a type of heart disease. It is also effective in preventing retinal degeneration, a disease of the eye. Although taurine-deficiency occurs primarily in cats, it may also occur in dogs, such as Cocker Spaniels, Golden Retrievers, Labrador Retrievers, Newfoundlands, Dalmatians, Portuguese Water Dogs, and English Bulldogs. Taurine may also be effective for general heart disease. Taurine and arachidonic acid are sourced from animal derived products or synthetic sources. For products that are considered “meat-free” or “animal free” (e.g. containing no animal products or by-products), the formula tends to be supplemented with taurine and arachidonic acid. Such foods, however, may still have one or more nutrients below the recommended levels and may present excessive micro or macro nutrients (e.g. fat, salt, sugar, etc.). As such, these food formulations may not be recommended for human or animals because such nutritional profiles may lead to health risks.

With all the limitations and challenges above comes the need for new formulations to address these limitations.

### SUMMARY

There remains a need for formulae that can be used in the development of food formulations and dietary supplements suitable for humans (adults and infants) and animals (including but not limited to fish and pets, including obligate carnivores).

It would be beneficial from a cost and environmental perspective to have a formulation with ingredients that provides the nutrients that companion animals need that are currently believed to be only present in animal-based or derived components or products. An aspect of the present invention is directed to a pet food composition with novel, natural ingredients that provide the required quantities of those nutrients, in addition to other essential nutrients with complete and balanced formulas that can meet dog and cat nutrient requirements. More specifically, an aspect of the present invention is a food formulation directed to a combination of one or more non-animal based or derived products such as algae, yeast, insects, plants or components thereof that meet the nutritional requirements of companion animals.

An aspect of the present invention is directed to pet food formulations that are not meat based (e.g. free of animal based or derived products, components or ingredients) but are suitable for pets including obligate carnivores. An aspect of the present invention is the use of algae in combination with other ingredients to provide food formulations that are not meat based. Another aspect of the present invention is directed to an animal free food composition comprising: (a) algae or algae-based products; (b) yeast or yeast -based products; (c) insect or insect-based products; (d) fungus or fungus-based products; and (e) plant or plant-based products.

Another aspect of the present invention is directed to the above noted food composition wherein the composition has nutritionally significant taurine content and nutritionally significant arachidonic content. Another aspect of the present invention is directed to the above noted food composition wherein the algae has a high hydrocolloid content >10% dry mass weight. Another aspect of the present invention is directed to the above noted food composition wherein the algae acts as an emulsifier, a stabilizer and/or a gelling agent.

Another aspect of the present invention is directed to the above noted food composition wherein the nutritionally significant taurine content is a taurine content over >10.1% on a dry weight basis. Another aspect of the present invention is directed to the above noted food composition wherein the nutritionally significant arachidonic is an arachidonic acid content over 0.005% on a dry weight basis. Another aspect of the present invention is directed to the above noted food composition further comprising vitamin B over 30 mg per 100 g by dry weight basis.

Another aspect of the present invention is directed to the above noted food composition wherein the algae or algae-based product is selected from the group consisting of Rhodophyta, Chlorophyta and combinations thereof. Another aspect of the present invention is directed to the above noted food composition wherein the algae or algae based product is selected from the group consisting of Nannochloropsis spp., Porphyridium spp. Palmaria spp., Parietochloris spp., Ceramium spp., Rodomella spp., Navicula spp., Pediastrum spp., Chondracanthus spp., Mazaella spp, Chondrus spp., Gelidium spp., Gracillaria spp., Grateloupia spp, Gloiopeltis spp., Porphyra spp., and Lobosphaera spp, Tetreselmis spp. Haematococcus, Ulva, Monostroma, Laminaria, Undaria, Hizikia, Himanthalia, Eisenia, Ascophyllum, Fucus and combinations thereof. Another aspect of the present invention is directed to the above noted food composition wherein the algae may be provided in the formulation in a concentration equal or above 0.5% per dry weight basis.

Yet another aspect of the present invention is directed to the above noted food composition wherein the insect is selected from the group consisting of Hermetia illucens (black soldier fly), Tenebrio molitor (mealworm), Rhinoceros beetles, Acheta domestics crickets and combinations thereof. Yet another aspect of the present invention is directed to the above noted food composition wherein the insect-based product is insect meal and insect oil. Yet another aspect of the present invention is directed to the above noted food composition wherein the concentration of the insect or insect based product is 2% to 50% per dry weight basis

Yet another aspect of the present invention is directed to the above noted food composition wherein the fungus is selected from the group consisting of Kluyveromyces marxianus, Aspergillus oryzae Fusarium venenatum, Geotrichum candidum, Penicillium camemberti, Penicillium roqueforti, Saccharomyces cerevisiae, and combination thereof. Yet another aspect of the present invention is directed to the above noted food composition wherein the concentration of the fungus or fungus based product is 2% to 50% per dry weight basis. Yet another aspect of the present invention is directed to the above noted food composition wherein the plant or plant based product is selected from the group consisting of peas, chickpeas, lentils, barley, sweet potatoes, pumpkin, flaxseed and combinations thereof.

Yet another aspect of the present invention is directed to the above noted food composition wherein the concentration of the plant or plant based products is 25% to 50% per dry weight basis. Yet another aspect of the present invention is directed to the above noted food composition further comprising one or more micronutrient based and/or vitamin based additives. Yet another aspect of the present invention is directed to the above noted food composition further comprising one or more micronutrient based and/or vitamin based product. Yet another aspect of the present invention is directed to the above noted food composition further comprising a palatant coating.

### BRIEF DESCRIPTION OF THE DRAWINGS

In the drawings, which illustrate embodiments of the invention:

FIGS. 1(a) and 1(b) illustrates preferred embodiments of the present invention.

### DETAILED DESCRIPTION OF THE PREFERRED EMBODIMENTS

The description that follows, and the embodiments described therein, is provided by way of illustration of an example, or examples, of particular embodiments of the principles and aspects of the present invention. These examples are provided for the purposes of explanation, and not of limitation, of those principles and of the invention.

It should also be appreciated that the present invention can be implemented in numerous ways, including as a process, method, an apparatus, a system, a device or a method. In this specification, these implementations, or any other form that the invention may take, may be referred to as processes. In general, the order of the steps of the disclosed processes may be altered within the scope of the invention. The description that follows, and the embodiments described therein, is provided by way of illustration of an example, or examples, of particular embodiments of the principles and aspects of the present invention. These examples are provided for the purposes of explanation, and not of limitation, of those principles and of the invention. It will be understood by a person skilled in the relevant art that in different geographical regions and jurisdictions these terms and definitions used herein may be given different names, but relate to the same respective systems, methods, compositions, etc.

Aspects of the present invention are directed to animal product free or meat-free food alternatives or formulations with natural source(s) of essential nutrients, such as taurine and arachidonic acid, that are sustainable and ethically produced and prevent nutrient deficiencies in pets, including obligate carnivores. An aspect of the present invention comprises food formulations, including pet food formulations, having (a) naturally sourced essential nutrients without using animal derived ingredients, components, products or by products; (b) balanced nutrient (e.g. amino acid, fatty acid, etc.) profile as defined by regulatory bodies or industry associations, including, but not limited to, the Association of American Feed Control Officials (“AAFCO”) and the regulatory body for the European pet food industry (e.g. FEDIAF); (c) nutritionally significant taurine content, (e.g. in a preferred embodiment, above 100 mg/100 g of food); and (d) nutritionally significant arachidonic content (e.g. in a preferred embodiment, above 6 mg/100 g of food).

A person skilled in the art will understand that an essential nutrient is a nutrient required for normal body functioning that cannot be synthesized in the required quantities by the organism. Categories of essential nutrient include, but are not limited to, vitamins, dietary minerals, essential fatty acids and essential amino acids

To achieve a nutritionally complete, animal product or by-product free food formulation, preferably pet food for dogs and cats, embodiments of the present invention are directed to a formulae based on one or more of the following: (a) algae and/or algae-based ingredient(s), which may provide key nutrients that are typically included in pet diets in the form of animal tissue/meat/by-products; (b) insects and/or insect products, (c) fungi and/or fungi products; (d) plant and/or plant products; and (e) bacteria and/or bacteria products. In a preferred embodiment, other components provide other macro and micronutrients needed for a complete diet that may not be provided (or provided in sufficient concentration) by (a) to (e). Macronutrients may include, but are limited to, protein content, amino acid profile, fat content, fatty acids and for example protein content and adequate amino acid profile. Complete and balanced pet food as well as nutritional adequacy is determined as meeting the requirements set out by FEDIAF, AAFCO, etc.

An aspect of the present invention is directed to formulations containing or having components or ingredients derived from macroalga (e.g. large aquatic photosynthetic plants that can be seen without the aid of a microscope) or microalgae (e.g. microscopic algae invisible to the naked eye). Macroalgae and microalgae include a number of different types of algae including green, red, brown and blue algae, which may grow in a variety of forms. The most familiar types can generally be divided into three groups: Green (Chlorophyta), Red (Rhodophyta), and Brown-Kelps (Phaeophyta). It will be understood that an aspect of the present invention would be directed to macroalga or microalge (or products derived therefrom, such as, for example, protein extracts, protein isolates, purified proteins, purified amino acids, lipid extracts, fatty acids, etc.) used in combination with any other ingredients to formulate pet food.

Another aspect of the invention are compositions directed to pet treats and pet supplements, as well as human food and supplements. Another aspect of the present invention is directed to food grade ingredients that can be added as a whole, or nutrients that can be extracted and then added to animal or human food products.

A preferred embodiment of the present invention relates to pet food formulae that provides key micro and macro nutrients and includes one or more of the following: (a) algae or algae-based or derived products, components or ingredients; (b) with yeast (or yeast -based products, components or ingredients); (c) insect (or insect-based products, components or ingredients); (d) fungus (or fungus-based products, components or ingredients); and/or (e) plant (or plant-based components, products or ingredients thereof). Key nutrients provided include one or more of the following: taurine, arachidonic acid and B vitamins, including folic acid.

In a preferred embodiment, the algae to be used in the present invention may have a high hydrocolloid content >10% dry mass weight. Preferably, the algae as an ingredient may act as an emulsifier, a stabilizer and/or a gelling agent. Algae with high hydrocolloid content could also be used as an emulsifier, a stabilizer and/or a gelling agent in products designated for human consumption. For human and animal (e.g. fish and pets) food products, algae can provide key essential nutrients including, but not limited to, taurine and arachidonic acid and act as an emulsifier, a stabilizer and/or a gelling agent particularly in animal free or meatless products.

In a preferred embodiment, at least one of the algae based component may be derived from algae having a high essential nutrient content (e.g. arachidonic acid content (e.g. over 0.005% on a dry weight basis), taurine content (e.g. over >0.1% on a dry weight basis), vitamin B content (e.g. over 30 mg per 100 g by dry weight basis)). In a preferred embodiment, the algae may be selected from the group consisting of the group Rhodophyta (also known as red algae) and Chlorophyta (also known as green algae). Some of the species in these groups include but are not limited to Nannochloropsis spp., Porphyridium spp. Palmaria spp., Parietochloris spp., Ceramium spp., Rodomella spp., Navicula spp., Pediastrum spp., Chondracanthus spp., Mazaella spp, Chondrus spp., Gelidium spp., Gracillaria spp., Grateloupia spp, Gloiopeltis spp., Porphyra spp., and Lobosphaera spp, Tetreselmis spp. Haematococcus, Ulva, Monostroma, Laminaria, Undaria, Hizikia, Himanthalia, Eisenia, Ascophyllum, Fucus. In a further preferred embodiment, the algae based product may be provided in the formulation in a concentration equal or above 0.5% per dry weight basis.

In a preferred embodiment, at least one of the insect based components may be derived from insects or insect-derived products such as, but not limited to, insect meal and insect oil. The foregoing may be derived from, but not limited to, the following species: Hermetia illucens (black soldier fly), Tenebrio molitor (mealworm), Rhinoceros beetles and Acheta domestics crickets. In a further preferred embodiment, the insect based product may be provided in the formulation in a concentration of 2% to 50% per dry weight basis.

In a preferred embodiment, at least one of the fungus based components may be derived from fungus such as, but not limited to, Kluyveromyces marxianus, Aspergillus oryzae Fusarium venenatum, Geotrichum candidum, Penicillium camemberti, Penicillium roqueforti, Saccharomyces cerevisiae. In a further preferred embodiment, the fungus based product may be provided in the formulation in a concentration of 2% to 50% per dry weight basis.

In a preferred embodiment, at least one of the plant-based components may be derived from plants such as, but are not limited to pea, chickpeas, lentils, barley, sweet potatoes, pumpkin, flaxseed. In a further preferred embodiment, the plant based product may be provided in the formulation in a concentration of 2% to 50% per dry weight basis.

In a preferred embodiment, the invention may also include one or more micronutrient based and/or vitamin based additives. In a further preferred embodiment, the algae based product may be provided in the formulation in a concentration of 2% to 50% per dry weight basis.

In a preferred embodiment, the invention may comprise one or more micronutrient based and/or vitamin based product. In a further preferred embodiment, the algae based product may be provided in the formulation in a concentration of 100%. In yet another preferred embodiment, food products of the present invention may be comprised 100% from natural ingredients or alternatively, food products of the present invention can include synthetic supplements added thereto.

In a preferred embodiment, there may also be provided a micronutrient mix: This mix includes any minerals, trace metals or vitamins that need to supplement in the product.

In yet another preferred embodiment, there may be provided a palatant coating: A palatant coating may be added to the pet food to improve palatability and nutrition. The coating can also contain one or more of the ingredients noted above. An preferred embodiment or example of such palatant is a mixture of red algae and insect oil; in this case the coating acts as a palatant due to the high palatability of insect oil but also contributes to the nutritional benefit of the food overall as red algae offers key nutrients that can be affected by processing parameters. Thus, adding red algae in the coating step increasing the availability of nutrients such as taurine and/or arachidonic acid and/or B vitamins.

In a preferred embodiment, the present invention can be meat-free and/or completely animal-free (100% vegan). It will be understood that “vegan” references products that are made without any animal products or by products. Meat free products may contain animal by products not considered as meat. This invention ensures all protein and fat nutrients are provided by the meat-free ingredients without the need of any supplements. In a preferred embodiment, the food formulae or formulation of the present invention may be applied to food that may be considered fresh or processed. Processed food includes air-dried, oven baked, canned, high temperature extrusion e.g. kibble and/or gently cooked as well as treats or snack foods for human and animals (including fish and pets).

In a preferred embodiment, the present invention may be directed to “toppers” (i.e. food items added to a meal to improve nutritional value or palatability) and/or/supplements for human and animal food to supply taurine and arachidonic acid. It will be understood by a person skilled in the relevant art that each supplement can be provided by separate organism. For example, one organism may product taurine while another organism provides arachidonic acid. One algal species (for example. Palmaria palmata) can provide taurine and a second algal species (for example, Porphyridium purpureum) can provide arachidonic acid.

In another preferred embodiment, the algae of the present invention can be used as whole biomass or as specific extracts thereof (e.g. oil extract, protein extract, free amino acid extract, taurine extract or arachidonic acid extract or a combination thereof)

In yet another preferred embodiment, the present invention is directed to human food, for both adults and infants. Taurine is recognized as a conditionally essential amino acid in adults. For infants it is an essential amino acid and added to infant formulas

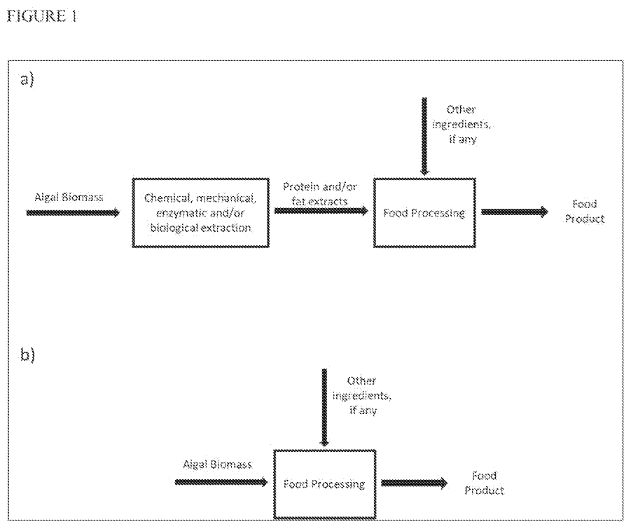

FIG. 1 shows a preferred embodiment of the present invention. As seen in FIG. 1(a), the algal biomass may be chemically, mechanically, enzymatically, or biologically extracted. The proteins and/or fats may then be extracted and afterward other ingredients may be added. However, extra processing of the algal biomass may not be required, as provided in FIG. 1(b).

### EXAMPLES

The following are embodiments or examples of meat-free and/or vegan formulas for cats on a dry mass basis that relate to this invention. Unless specified as “protein” or “extract”, ingredients are whole dry biomass:

| Meat Free Cat Food Formula | % |
|---|---|
| Insect Protein (from Hermetia illucens) | 29.0 |
| Green algae (Arthrospira spp.) | 20.0 |
| Red algae (Gracilaria spp.) | 20.0 |
| Insect oil (from Hermetia illucens) | 5.0 |
| Yeast (Saccharomyces spp.) | 10.0 |
| Chickpeas | 8.0 |
| Peas | 5.0 |
| Micronutrient Mix | 2.0 |

| Meat Free Cat Food Formula 2 | % |
|---|---|
| Insect Protein (from Tenebrio molitor) | 40.0 |
| Yeast (Saccharomyces spp.) | 12.0 |
| Red algae (Gracilaria spp.) | 10.0 |
| Peas | 10.0 |
| Chickpeas | 10.0 |
| Insect oil (from Tenebrio molitor) | 8.0 |
| Kelp (Laminaria spp.) | 5.0 |
| Oats | 3.0 |

| Vegan Cat Food Formula | % |
|---|---|
| Green Algae 1 (Arthrospira spp.) | 33.0 |
| Red Algae (Mazzaella spp.) | 20.0 |
| Yeast (Kluyveromyces spp.) | 15.0 |
| Flaxseed | 12.0 |
| Lentils | 8.0 |
| Green Algae 2 (Lobosphaera spp.) | 8.0 |
| Chickpeas | 2.0 |
| Micronutrient mix | 2.0 |

Cats are companion animals that are typically known or considered to be “obligate carnivores”, which means that they require nutrients that are supplied through tissue, fats and meat from traditional livestock. This invention supports, that in fact, cats are not obligate carnivores. They do require taurine, arachidonic acid and B vitamins in their diet which have been traditionally sourced from livestock products; however, these nutrients can be sourced non-animal sources, such as, for example, from algae. While dogs are not recognized as “obligate carnivores”, evidence suggests that taurine, arachidonic acid, a B vitamin supplementation is needed in their diets. Therefore, dog's meat-free and vegan diets may need formulae such as the ones in this invention. Example of dogs' meat-free and vegan diet are found below

| Meat-Free Dog Food | % |
|---|---|
| Insect protein (Hermetia illucens) | 19.0 |
| Yeast (Saccharomyces spp.) | 15.0 |
| Lentils | 15.0 |
| Chickpeas | 11.0 |
| Oats | 10.0 |
| Insect Larvae (Hermetia illucens) | 9.5 |
| Red algae (Graciliaria spp.) | 5.0 |
| Insect Oil (Hermetia illucens) | 4.1 |
| Sweet Potato | 4.0 |
| Green algae (Ulva spp.) | 2.9 |
| Beet Pulp | 2.5 |

| Vegan Dog Food Formula | % |
|---|---|
| Green Algae (Chlorella spp.) | 14.0 |
| Yeast (Saccharomyces spp.) | 14.0 |
| Chickpea | 13.0 |
| Lentils | 10.0 |
| Oats | 10.0 |
| Rapeseed | 10.0 |
| Sweet Potato | 8.0 |
| Red algae (Mazzaella spp.) | 5.0 |
| Sunflower seeds | 5.0 |

Preferred embodiments of the present invention can be implemented in numerous configurations depending on implementation choices based upon the principles described herein. Various specific aspects are disclosed, which are illustrative embodiments not to be construed as limiting the scope of the disclosure. Although the present specification describes components and functions implemented in the embodiments with reference to standards and protocols known to a person skilled in the art, the present disclosures as well as the embodiments of the present invention are not limited to any specific standard or protocol.

1. An animal free food composition comprising:
(a) algae or algae-based products;
(b) yeast or yeast -based products;
(c) insect or insect-based products;
(d) fungus or fungus-based products; and
(e) plant or plant-based products.

2. The food composition of claim 1 wherein the composition has nutritionally significant taurine content and nutritionally significant arachidonic content.

3. The food composition of claim 2 wherein the algae has a high hydrocolloid content >10% dry mass weight.

4. The food composition of claim 3 wherein the algae acts as an emulsifier, a stabilizer and/or a gelling agent.

5. The food composition of claim 4 wherein the nutritionally significant taurine content is a taurine content over >10.1% on a dry weight basis.

6. The food composition of claim 5 wherein the nutritionally significant arachidonic is an arachidonic acid content over 0.005% on a dry weight basis.

7. The food composition of claim 6 further comprising vitamin B over 30 mg per 100 g by dry weight basis.

8. The food composition of claim 7 wherein the algae or algae-based product is selected from the group consisting of Rhodophyta, Chlorophyta and combinations thereof.

9. The food composition of claim 8 wherein the algae or algae based product is selected from the group consisting of Nannochloropsis spp., Porphyridium spp. Palmaria spp., Parietochloris spp., Ceramium spp., Rodomella spp., Navicula spp., Pediastrum spp., Chondracanthus spp., Mazaella spp, Chondrus spp., Gelidium spp., Gracillaria spp., Grateloupia spp, Gloiopeltis spp., Porphyra spp., and Lobosphaera spp, Tetreselmis spp. Haematococcus, Ulva, Monostroma, Laminaria, Undaria, Hizikia, Himanthalia, Eisenia, Ascophyllum, Fucus and combinations thereof,.

10. The food composition of claim 9 wherein the algae may be provided in the formulation in a concentration equal or above 0.5% per dry weight basis.

11. The food composition of claim 10 wherein the insect is selected from the group consisting of Hermetia illucens (black soldier fly), Tenebrio molitor (mealworm), Rhinoceros beetles, Acheta domestics crickets and combinations thereof.

12. The food composition of claim 11 wherein the insect-based product is insect meal and insect oil.

13. The food composition of claim 12 wherein the concentration of the insect or insect based product is 2% to 50% per dry weight basis.

14. The food composition of claim 13 wherein the fungus is selected from the group consisting of Kluyveromyces marxianus, Aspergillus oryzae Fusarium venenatum, Geotrichum candidum, Penicillium camemberti, Penicillium roqueforti, Saccharomyces cerevisiae, and combination thereof.

15. The food composition of claim 14 wherein the concentration of the fungus or fungus based product is 2% to 50% per dry weight basis.

16. The food composition of claim 15 wherein the plant or plant based product is selected from the group consisting of peas, chickpeas, lentils, barley, sweet potatoes, pumpkin, flaxseed and combinations thereof.

17. The food composition of claim 16 wherein the concentration of the plant or plant based products is 25% to 50% per dry weight basis.

18. The food composition of claim 17 further comprising one or more micronutrient based and/or vitamin based additives.

19. The food composition of claim 18 further comprising one or more micronutrient based and/or vitamin based product.

20. The food composition of claim 19 further comprising a palatant coating.
